(19) United States
(12) Patent Application Publication
(10) Pub. No.: US 2004/0143262 A1
(43) Pub. Date: Jul. 22, 2004

(54) Surgical perforation device and method with pressure monitoring and staining abilities

(75) Inventors: Naheed Visram, Markham (CA); Krishan Shah, Mississauga (CA); Amanda April Hartley, Brampton (CA)

Correspondence Address: OGILVY RENAULT, 1981 MCGILL COLLEGE AVENUE, SUITE 1600, MONTREAL, QC, H3A2Y3, CA

(73) Assignee: Baylis Medical Company Inc., Montreal (CA)

(21) Appl. No.: 10/666,288

(22) Filed: Sep. 19, 2003

Related U.S. Application Data
(63) Continuation-in-part of application No. 10/347,366, filed on Jan. 21, 2003.

(51) Int. Cl. A61B 18/14

(52) U.S. Cl. 606/45; 600/561

(57) ABSTRACT

A device with a functional tip containing at least one active electrode capable of creating a controlled perforation in body tissue through the application of Radio Frequency (RF) energy is described. The position of the tip of the device can be determined in response to pressure sensed at the tip and determined by a monitor. The device is useful to remove or perforate unwanted tissue in a controlled manner in any location in the body, particularly in the atrial septum for controlled transseptal puncture. In this application, the device is introduced into the right atrium, and the functional tip is then positioned against the atrial septum. Energy is applied to create the perforation and pressure is monitored to determine if the perforation was created in a desired location. Other possible applications include the removal of plaque or thrombotic occlusions from diseased vessels.

### REFERENCE TO PRIOR APPLICATION

This is a continuation-in-part application of application Ser. No. 10/347,366 filed Jan. 21, 2003.

### TECHNICAL FIELD

The invention relates to a surgical perforation device and method with pressure monitoring and staining abilities. More specifically, the invention relates to a device and method for staining the atrial septum, creating a controlled perforation in the atrial septum while monitoring blood pressure and delivering a dilator and guiding sheath to the left atrium through the perforation over the surgical device.

### BACKGROUND OF THE ART

Electrosurgical devices perforate or cut tissues when radio frequency (RF) electrical energy rapidly increases tissue temperature to the extent that the intracellular fluid becomes converted to steam, inducing cell lysis as a result of elevated pressure within the cell. The radio frequency range lies between 10 kHz and 300 MHz, but electrosurgical devices usually operate at a frequency between 400 kHz and 550 kHz. This technology can be used to create perforations in different types of tissue, such as heart tissue, vascular occlusions, and others. Commonly, RF devices are described for use in perforating vascular occlusions. A device to dilate and/or lance blood vessels that are morbidly contracted or clogged is described in European Patent Application Number EP 0315730, of Osypka, published May 15, 1989. This device describes the use of RF energy in either bipolar or monopolar application modes to open blood vessels by means of heat. Other devices intended to use RF energy to pass through occluded vessels have also been described (U.S. Pat. No. 5,364,393, of Auth et al., issued Nov. 15, 1994, WO 93/20747, publication of PCT Patent Application No. PCT/US93/03759, of Rosar, published Oct. 28, 1993, U.S. Pat. No. 5,098,431, of Rydell, issued Mar. 24, 1992, and U.S. Pat. No. 4,682,596 of Bales et al., issued Jul. 28, 1987). U.S. Pat. No. 6,293,945 B1, of Parins et al., issued Sep. 25, 2001 describes an electrosurgical instrument with suction capability. This device has three functions at the tip including cutting, coagulating, and suction. None of these devices however incorporate a means for verifying the location of the device within the body. One means for verifying location is described in U.S. Pat. No. 4,936,281, of Stasz, issued Jun. 26, 1990, which describes an ultrasonically enhanced RF catheter used for cutting. An ultrasonic transducer connected to an electronics module receives echo signals, enabling Doppler flow readings and ultrasound imaging of the vessel.

Having reliable information about the location of electrosurgical devices within a body is an important aid to performing a successful procedure. It is often valuable to have more than one source of this information because every imaging technique has limitations, and using only one method can lead to erroneous information. Relative blood pressure measurements can be a useful tool to verify the position of a device in a body. Different locations in the body are known to have characteristic blood pressure ranges. Knowing the blood pressure at the tip of a perforation device is a useful tool to determine the location of the device, particularly in instances where imaging techniques provide inconclusive information. A device that is used for measuring pressure in coronary arteries is described in U.S. Pat. No. 4,928,693, of Goodin et al., issued May 29, 1990; however the device is not capable of perforating tissue using RF energy. U.S. Pat. No. 6,296,615 B1, of Brockway et al., issued Oct. 2, 2001, describes a catheter with a physiological sensor. This catheter consists of a pressure transducer for monitoring pressure, as well as the ability to detect and/or transmit an electrical signal.

It is often required to create a perforation in the atrial septum to gain access to the left side of the heart interventionally to study or treat electrical or morphological abnormalities. It is also often desirable to create a hole in the septum in order to shunt the blood flow between the left and right sides of the heart to relieve high pressure or provide more blood flow to certain areas. Historically in these instances, a dilator and guiding sheath are introduced into the femoral vein over a guidewire and advanced into the right atrium. The guidewire, dilator and guiding sheath are usually packaged as a kit with the guiding sheath designed to track over the dilator. In most designs, the distal end of the dilator extends out typically 4 cm (about 1.57″) beyond the distal end of the sheath once the two devices are locked together. Once the dilator and guiding sheath are positioned appropriately in the right atrium, a stiff needle such as the Transseptal needle of Cook Incorporated, Bloomington, Ind., USA is introduced through the dilator and guiding sheath set in the femoral vein and advanced through the vasculature into the right atrium. From there the needle tip is positioned at the fossa ovalis, the preferred location on the septum for creating a hole. Once in position, mechanical energy is used to advance the needle through the septum and into the left atrium. Once in the left atrium the needle can be attached to a pressure transducer and the operator can confirm a left atrial pressure before continuing with the procedure. An operator may dilate the hole by advancing the dilator over the needle into the left atrium and tracking the guiding sheath over the dilator and into the left atrium to provide access for other devices to the left heart once the needle and dilator are removed. As well, the operator may use another device such as a balloon catheter delivered over a guidewire to enlarge the hole made by the needle if a shunt between the right and left atria is desired.

Another device and method for creating a transseptal puncture is described in U.S. Pat. No. 5,403,338, of Milo, issued Apr. 4, 1995, which describes a punch that is intended to create an opening between two compartments. This device also makes use of mechanical energy, as with the transseptal needle.

These methods of creating a transseptal perforation rely on the skill of the operator and require practice to be performed successfully. The needles used in this procedure are very stiff and can damage the vessel walls as they are being advanced. In addition, the amount of force required to perforate the septum varies with each patient. If too much force is applied there is the possibility of perforating the septum and continuing to advance the needle so far that damage is done to other areas of the heart. C. R. Conti (1993) discusses this possibility, and states that if the operator is not careful, the posterior wall of the heart can be punctured by the needle when it crosses the atrial septum because of the proximity of the two structures. It can also be difficult to position the needle appropriately in hearts that have malformations, or an a typical orientation. Justino et al. (2001) note that despite improvements to the technique with the needle since its first introduction, most large series continue to report failed or complicated mechanical transseptal punctures, for reasons such as unusual septal thickness, or contour. Patients with a muscular septum, as well as those with a thick septum can benefit from an alternative to the transseptal needle puncture (Benson et al, 2002), as it is difficult to control the amount of mechanical force required to create the puncture. Furthermore, children born with heart defects such as hypoplastic left heart syndrome could benefit from an alternative technique. The abnormal anatomy of these patients including a small left atrium increases the likelihood of injury or laceration of surrounding structures during transseptal puncture (Sarvaas, 2002). The patient population discussed above would benefit from a device and technique for transseptal puncture that allows for a more controlled method of perforation and a method to confirm that the perforation has been made in the correct location.

### SUMMARY OF THE INVENTION

The present invention provides a surgical perforation device with pressure monitoring and optionally, staining abilities and a method therefor.

In accordance with a first aspect of the invention, there is provided a surgical device for cutting material and monitoring pressure. The surgical device comprises an elongate member having a distal region and a proximal region; an energy delivery device associated with the elongate member at the distal region for delivering cutting energy to the material, said energy delivery device adapted for connection to an energy source; and a pressure sensing mechanism associated with the distal region for monitoring pressure about the distal region.

The cutting energy is at least one form of energy selected from a group consisting of: electrical current; microwave; ultrasound; and laser. When the energy is electrical current, the current may have a frequency within the radio frequency (RF) range. Further, when the material to be cut comprises cellular tissue, the energy delivery device is operable to deliver sufficient energy to the tissue to result in a rapid increase in the intracellular temperature causing vaporization of intracellular water and subsequent cell lysis.

In accordance with an embodiment of the first aspect, the pressure sensing mechanism comprises a pressure transmitting lumen extending between the proximal and distal regions. The lumen at the proximal region is adapted for fluid communication with a pressure transducer that provides a signal which varies as a function of pressure. It may be further for fluid communication with an environment about said distal region. In such an embodiment, the distal region may comprise at least one opening to the environment and the lumen is in fluid communication with the at least one opening. When the distal region comprises multiple openings to the environment in fluid communication with the lumen, preferably, at least some of the multiple openings are located distally and some of the multiple openings are located proximally with respect to each other and the some of the openings located distally are larger than the some of the openings located proximally. Preferably, the lumen is adapted for injecting a fluid through the one or more openings, for example, to stain a region of material.

In accordance with a further embodiment of the first aspect, the pressure sensing mechanism comprises a pressure transducer on-board the elongate member and associated with the distal region. The transducer is adapted for communication with a pressure monitoring system.

The energy delivery device may comprise a functional tip with at least one active electrode. Further the energy delivery device may comprise a functional tip having two or more electrodes and the electrodes may be configured in an arrangement where at least one of the electrodes is active and at least one is a return electrode.

optionally, the device may comprise at least one depth marking. Further, the may comprise at least one radiopaque marker. As well, the distal region of the device may be radiopaque.

In accordance with a further aspect of the invention, there is provided a method of surgery. The method comprises: (i) introducing a surgical device into a body of a patient, the surgical device comprising an elongate member having a distal region and a proximal region, an energy delivery device proximate to the distal region capable of cutting material and a pressure sensing mechanism for determining pressure in the body proximate to the distal region; (ii) positioning the energy delivery device to a first desired location in the patient's body adjacent material to be cut; (iii) delivering energy using the energy delivery device to cut said material; and (iv) measuring pressure in the body using the pressure sensing mechanism in order to determine the position of the surgical device at least one of before and after step (iii). In a preferred embodiment of this aspect, step (ii) comprises staining a region of tissue in the first desired location in the patient's body.

The method may further comprise a step of (v) advancing the device to a second desired location. In an embodiment of this aspect, the surgical device comprises at least one depth marking and at least one radiopaque marker and step (v) comprises monitoring at least one of said depth markings and at least one of said radiopaque markers. The method may comprise a step of: (vi) measuring pressure using the pressure sensing mechanism at the second location. The surgical device may comprise at least one depth marking and at least one radiopaque marker and step (vi) may be performed after confirming the position of the pressure sensing mechanism at the second location using at least one of said depth markings and said radiopaque markers.

As a feature of this method aspect, step (i) comprises introducing the device into the patient's vasculature. The step of introducing the device into the patient's vasculature may comprise inserting the device into a dilator and a guiding sheath positioned in the patient's vasculature. Optionally, the device and at least one of the dilator and sheath comprise a radiopaque marking and step (ii) may comprise aligning the radiopaque markings to aid in positioning the device. The method may further comprise a step of (v) advancing the dilator and the sheath into the second location together over the spatially fixed surgical device or (v) advancing the dilator, sheath and surgical device all together into the second location.

In accordance with the method, the material may be tissue located on an atrial septum of a heart. Further, the region of tissue to be stained may be the fossa ovalis of a heart. In such a case, the pressure measured at the second location is the blood pressure in the left atrium.

In another aspect of the invention, there is provided an electrosurgical device. The electrosurgical device comprises a elongate member having a distal region and a proximal region, said distal region insertable within and along a lumen within a body of a patient and maneuverable therethrough to a desired location where the device is operated to cut material and monitor pressure at the desired location; at least one electrode associated with the distal region for cutting tissue, said at least one electrode adapted for coupling to an electrical power source; and a pressure sensing mechanism associated with the distal region for sensing pressure at the desired location within the body, said mechanism adapted for coupling to a pressure monitoring system.

Preferably, the pressure sensing mechanism is configured to minimize a portion of the elongate member that is necessary to be located at the desired location to monitor pressure.

In a further aspect, there is provided a surgical device comprising means for cutting material at a desired location in a body of a patient; and means for determining a position of the device responsive to pressure within the body.

As a feature of this aspect, the device comprises a flexible elongate member having a proximal region and a distal region, the distal region is adapted for insertion within and along a lumen within the body and maneuverable therethrough to the desired location and the means for determining a position of the device is operable to determine the position of the distal region.

In accordance with yet another feature there is provided a method of cutting tissue at a desired location in a body of a patient. The method comprises: inserting a surgical device into the body, said surgical device comprising means for cutting material and means for determining a position of the device responsive to pressure within the body; and positioning said surgical device at the desired location in response to the means for determining a position of the device.

The method may comprise cutting material at the desired location and further comprise advancing the device in the body in response to said means for determining a position of the device. Optionally, the method comprises re-positioning said device for re-cutting in response to said means for determining a position of the device.

It is to be understood that references to cut or cutting material such as tissue in relation to the present invention include perforating, ablating, coagulating and removing material.

### BRIEF DESCRIPTION OF THE DRAWINGS

In order that the invention may be readily understood, embodiments of the invention are illustrated by way of examples in the accompanying drawings, in which:

FIG. 1 illustrates a schematic view of an electrosurgical system including a preferred embodiment of an electrosurgical device in accordance with a preferred embodiment of the invention;

FIG. 2 illustrates a side cross-sectional view of the device of FIG. 1;

FIG. 3 illustrates a cross-sectional view of an alternate embodiment of the device;

FIG. 4 illustrates an active electrode of the device of FIG. 1;

FIG. 5 illustrates an alternate embodiment of the distal region of a device in accordance with the invention;

FIG. 6 illustrates a side cross-sectional view of an alternate embodiment of the device;

FIG. 7 illustrates a first position of the device of FIG. 1 against an atrial septum of a heart;

FIG. 8 illustrates a second position of the device of FIG. 1, after successful perforation of the atrial septum; and

FIGS. 9A and 9B illustrate a flow chart of a transseptal perforation method in accordance with this invention.

It will be noted that throughout the appended drawings, like features are identified by like reference numerals.

### DETAILED DESCRIPTION OF THE INVENTION

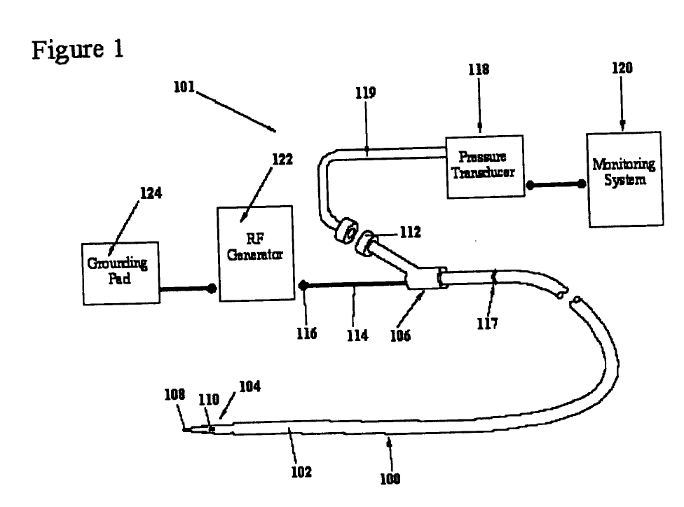

FIG. 1 illustrates a preferred embodiment of an electrosurgical perforation device 100 in accordance with the invention in an electrosurgical system 101. Device 100 comprises an elongate member 102 having a distal region 104, and a proximal region 106. Distal region 104 is adapted to be inserted within and along a lumen of a body of a patient, such as a patient's vasculature, and maneuverable therethrough to a desired location proximate to material such as tissue to be cut.

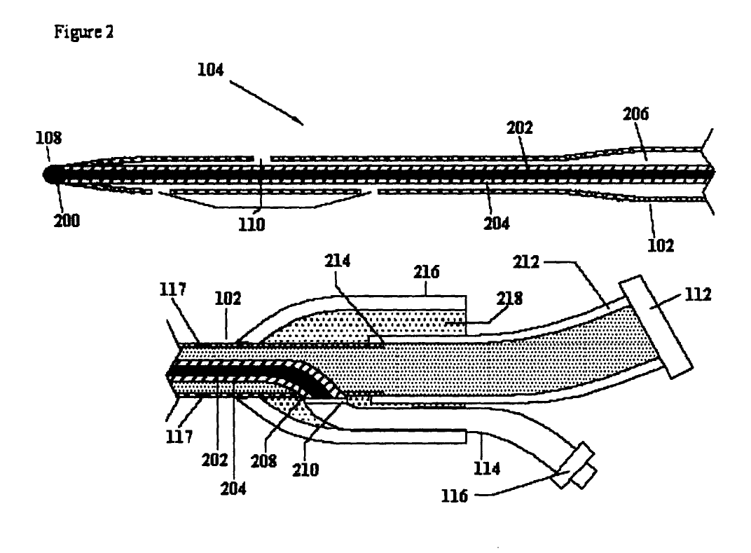

The elongate member 102 is typically tubular in configuration, having at least one lumen extending from proximal region 106 to distal region 104 such as lumen 206 shown in FIG. 2. Elongate member 102 is preferably constructed of a biocompatible polymer material that provides column strength to device 100. The elongate member 102 is sufficiently stiff to permit a dilator 704 and a soft guiding sheath 702 to be easily advanced over device 100 and through a perforation. Examples of suitable materials for the tubular portion of elongate member 102 are polyetheretherketone (PEEK), and polyimide. In a preferred embodiment, the outer diameter of the tubular portion of elongate member 102 tapers down to connect to distal region 104. In alternate embodiments the outer diameter of elongate member 102 and the outer diameter of distal region 104 are the same.

Distal region 104 is constructed of a softer polymer material so that it is pliable and atraumatic when advanced through vasculature. An example of a suitable plastic is Pebax (a registered trademark of Atofina Chemicals, Inc.). Distal region 104 preferably has a smaller outer diameter than elongate member 102 so that dilation of a perforation is limited while the distal region 104 is advanced through the perforation. Limiting dilation ensures that the perforation will not cause hemodynamic instability once device 100 is removed. The outer diameter of distal region 104 will preferably be no larger than 0.035° (0.897 mm). This is comparable to the distal outer diameter of the transseptal needle that is traditionally used for creating a perforation in the atrial septum. Elongate member 102 is preferably no larger than 0.050″ (1.282 mm) which is also comparable to the transseptal needle dimensions.

Distal region 104 comprises an energy delivery device configured as a functional tip 108. Functional tip 108 comprises at least one active electrode made of a conductive and radiopaque material, such as stainless steel, tungsten, platinum, or another metal. One or more radiopaque markings (not shown) may be affixed to elongate member 102 to highlight the location of the transition from distal region 104 to elongate member 102, or other important landmarks on device 100. Alternately, the entire distal region 104 of device 100 may be radiopaque. This can be achieved by filling the polymer material, Pebax used to construct distal region 104 with a radiopaque filler. An example of a suitable radiopaque filler is Bismuth. Distal region 104 defines at least one opening 110 in fluid communication with main lumen 206 (FIG. 2) as described further below.

Proximal region 106 comprises a hub 112, a cable 114, and a connector 116. Proximal region 106 may also have one or more depth markings 117 to indicate distances from functional tip 108, or other important landmarks on device 100. Hub 112 is configured to releaseably couple device 100 to an external pressure transducer 118 via external tubing 119. External pressure transducer 118 is coupled to a monitoring system 120 that converts a pressure signal from external pressure transducer 118 and displays pressure as a function of time. Cable 114 is coupled to connector 116 which is used to releaseably couple the device 100 to an energy source such as a generator 122.

Generator 122 is preferably a radiofrequency (RF) electrical generator that is designed to work in a high impedance range. Because of the small size of functional tip 108 the impedance encountered during RF energy application is very high. General electrosurgical generators are typically not designed to deliver energy in these impedance ranges, so only certain RF generators can be used with this device. In the preferred embodiment, the energy is delivered as a continuous wave at a frequency between about 400 kHz and about 550 kHz. An appropriate generator for this application is the BMC RF Perforation Generator (model number RFP-100, Baylis Medical Company, Montreal, Canada). This generator delivers continuous RF energy at about 460 kHz. A grounding pad 124 is coupled to generator 122 for attaching to a patient to provide a return path for the RF energy. Other embodiments could use pulsed or non-continuous RF energy. In still other embodiments of the electrosurgical perforation device 100, different energy sources may be used, such as microwave, ultrasound, and laser with appropriate energy delivery coupling devices and energy delivery devices.

Referring to FIG. 2 a cross-section of device 100 is illustrated in accordance with the embodiment of FIG. 1. Functional tip 108 comprises an active electrode 200 that is coupled to an insulated conducting wire 202. Conducting wire 202 is preferably attached to distal region 104 using an adhesive. Alternately, distal region 104 is melted onto insulation 204 on conducting wire 202 to form a bond.

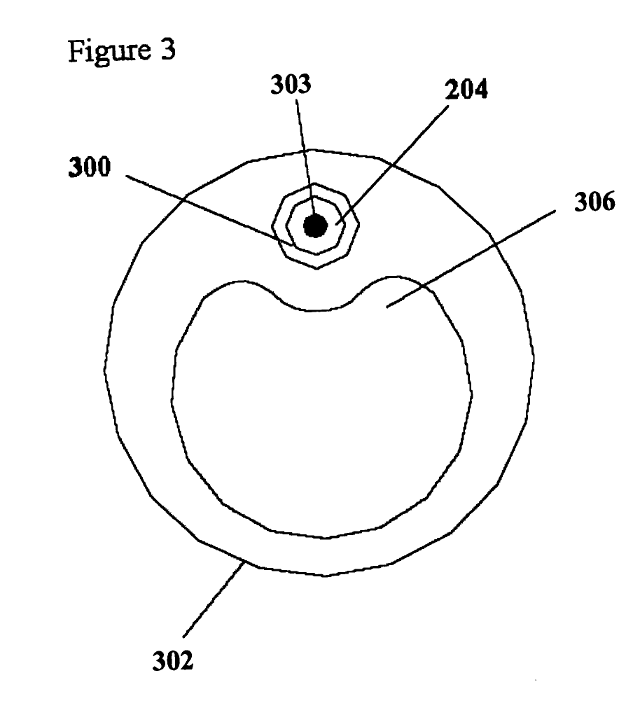

Conducting wire 202 carries electrical energy from generator 122 to the active electrode 200. Conducting wire 202 is covered with electrical insulation 204 made of a biocompatible material that is able to withstand high temperatures such as polytetrafluoroethylene (PTFE), or other insulating material. Conducting wire 202 preferably extends through a main lumen 206 of device 100 which lumen extends from proximal region 106 to distal region 104. In an alternate embodiment shown in cross section view in FIG. 3, an elongate member 302 comprises main lumen 306 and a separate lumen 300. The separate lumen 300 contains a conducting wire 303 therein and main lumen 306 is used for aspiration of blood and injection of contrast and other media. This embodiment of elongate member 302 allows a dedicated lumen for each function of device 100.

In the preferred embodiment of FIG. 2, main lumen 206 extends from proximal region 106 along elongate member 102 and through distal region 104 of device 100. At least one opening 110 at the distal region 104 provides a pathway between main lumen 206 and the environment surrounding distal region 104, such as a desired location within a patient's body. Openings 110 are sufficiently dimensioned to easily aspirate blood to and through main lumen 206 and to inject radiopaque contrast; however, openings 110 are limited in number and dimension so that they do not compromise the structural integrity of distal region 104. In order to facilitate even distribution of contrast agent and to prevent pooling in main lumen 206 at distal region 104, openings 110 may be dimensioned such that distally located openings (not shown) are larger than proximally located openings (not shown). The location of openings 110 is as close to functional tip 108 as possible so that only a small portion of device 100 is required to be proximate to the desired location for the determination of pressure.

Hub 112 is configured for releaseably coupling to an external pressure transducer 118, or a standard syringe. Preferably, hub 112 comprises a female Luer lock connection. Hub 112 is coupled to main lumen 206 via tubing 212 to provide a pathway from main lumen 206 to external pressure transducer 118 so that blood pressure can be determined using a method that is known to those of ordinary skill in the art. Conducting wire 202 exits elongate member 102 through an exit point 208. Exit point 208 is sealed with an adhesive or a polymeric material. Conducting wire 202 is electrically coupled to cable 114 by a joint 210. This joint can be made by soldering, or another wire joining method known to people of ordinary skill in the art. Cable 114 terminates with a connector 116 that can mate with either the generator 122, or a separate extension connector cable (not shown). Cable 114 and connector 116 are made of materials suitable for sterilization, and will insulate the user from energy traveling through the conductor.

Elongate member 102 is coupled to tubing 212 at proximal end 214 of elongate member 102. Tubing is made of a polymeric material that is more flexible than elongate member 102. A suitable material for tubing is polyvinylchloride (PVC), or another flexible polymer. Tubing 212 is coupled to hub 112. This configuration provides a flexible region for the user to handle when releaseably coupling external pressure transducer 118, or other devices to hub 112. Couplings between elongate member 102 and tubing 212, and tubing 212 and hub 112 are made with an adhesive such as a UV curable adhesive, an epoxy, or another type of adhesive.

A housing 216 surrounds joint 210 and proximal end of elongate member 102 in order to conceal these connections. Housing is made of a polymeric material, and is filled with a filling agent 218 such as an epoxy, or another polymeric material in order to hold cable 114 and tubing 212 in place.

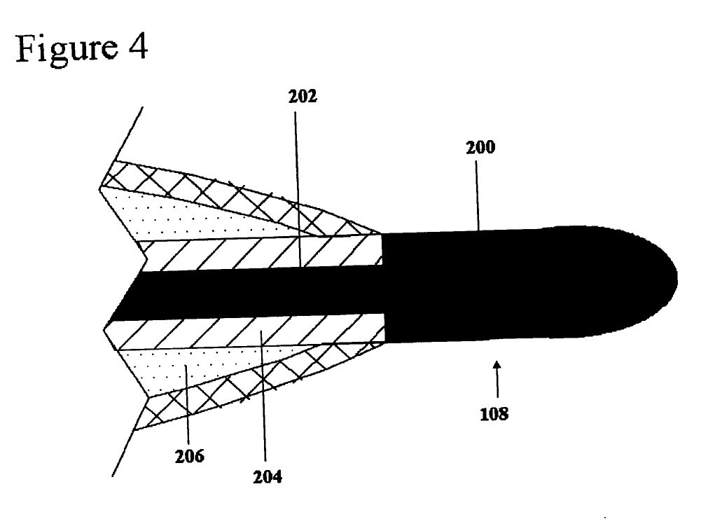

Referring to FIG. 4 there is illustrated a view of a preferred embodiment of functional tip 108. Functional tip 108 comprises one active electrode 200 configured in a bullet shape. Active electrode 200 is preferably 0.059″ (0.15 cm) long and preferably has an outer diameter of 0.016″ (0.04 cm). Active electrode 200 is coupled to an end of conducting wire 202, also made out of a conductive and radiopaque material. RF energy is delivered through active electrode 200 to tissue, and travels through the patient to grounding pad 124, which is connected to generator 122. Alternate embodiments of active electrode 200 are configured in shapes other than a bullet. These shapes include a spherical shape, a rounded shape, a ring shape, a semi-annular shape, an ellipsoid shape, an arrowhead shape, a spring shape, a cylindrical shape, among others.

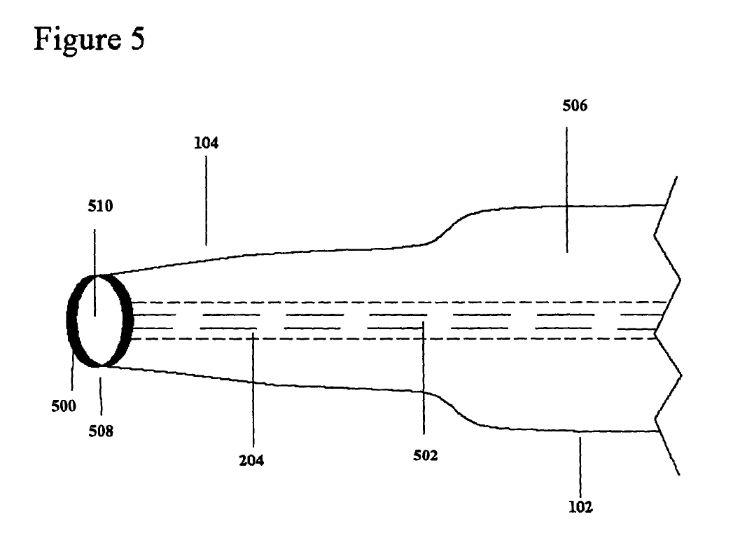

Referring to FIG. 5 there is illustrated an alternate embodiment of a functional tip 508. Functional tip 508 comprises one active electrode 500 in a ring configuration. Conducting wire 502 is coupled to the active electrode 500, and active electrode 500 is positioned around a perimeter of a single opening 510 that provides a pathway between main lumen 506 and a patient's body. Another similar embodiment to functional tip 108 comprises an active electrode in a partially annular shape (not shown). In other embodiments (not shown), a functional tip comprises multiple electrodes. Such electrodes may operate in a monopolar mode as with the embodiments detailed in FIGS. 2 and 5. Otherwise, such electrodes are arranged such that the RF energy is delivered through at least one active electrode at the functional tip, and returns to the generator through at least one return electrode at the functional tip. The use of an active and a passive electrode attached to device 100 eliminates the need for a grounding pad 124 to be attached to the patient as is well understood by persons of ordinary skill in the art.

In the preferred embodiment, external pressure transducer 118 is releaseably coupled to device 100. Hub 112 is coupled to external tubing 119 that is coupled to external pressure transducer 118 as shown in FIG. 1. External tubing 119 is flushed with saline to remove air bubbles. When device 100 is positioned in a blood vessel in a body, pressure of fluid at distal region 104 exerts pressure through openings 110 on fluid within main lumen 206, which exerts pressure on saline in external tubing 119, which exerts pressure on external pressure transducer 118. The at least one opening 110 and lumen 206 provide a pressure sensing mechanism in the form of a pressure transmitting lumen for coupling to pressure transducer 118. External pressure transducer 118 produces a signal that varies as a function of the pressure it senses. External pressure transducer 118 is also releaseably electrically coupled to a pressure monitoring system 120 that converts the transducer's signal and displays a pressure contour as a function of time.

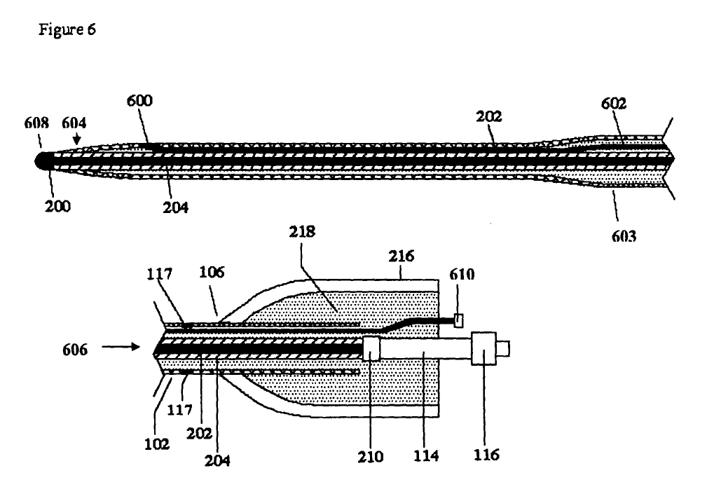

Referring to FIG. 6 there is illustrated a side cross-sectional view of proximal 606 and distal 604 regions of an alternate embodiment of an electrosurgical perforation device that does not use an external pressure transducer. In this embodiment the pressure sensing mechanism comprises an on-board pressure transducer 600 coupled by an adhesive to elongate member 603 at distal region 604. The pressure transducer 600 is configured at distal region 604 such that pressure close to functional tip 608 can be transduced. The on-board pressure transducer 600 is electrically coupled to a pressure communicating cable 602 to provide power to transducer 600 and to carry a pressure signal to proximal region 606 of the electrosurgical perforation device. Pressure communicating cable 602 terminates in a monitoring system connector 610 that is configured to be releaseably coupled to pressure monitoring system 120. Monitoring system 120 converts the pressure signal and displays pressure as a function of time. In the embodiment of FIG. 6, a main lumen such as the main lumen 206 of FIG. 2 is not required for fluid communication with an external pressure transducer 118. In addition, this embodiment does not require openings, such as openings 110 of FIG. 2, at distal region 606 for fluid communication with a main lumen. However, a lumen with openings may be provided for injecting or aspirating fluids, if desired.

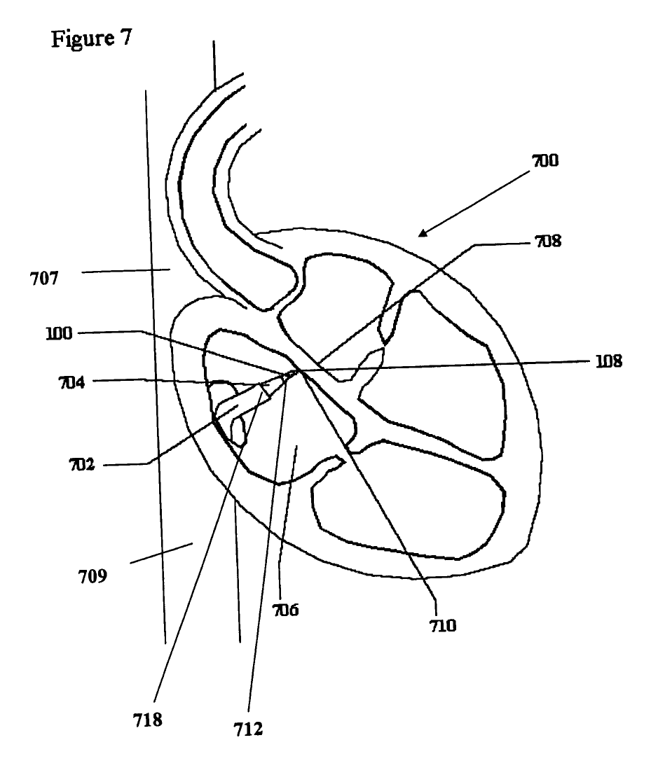

Device 100 of this invention or alternate embodiments can be used for general electrosurgery in instances where it is desirable to cut tissue or other material and simultaneously determine fluid pressure. More specifically, it can be used for creating a perforation such as a transseptal perforation. In order to create a perforation in the atrial septum to gain access to the left side of the heart the electrosurgical perforation device 100 is delivered to the atrial septum (FIG. 7). This is most commonly done using a dilator 704 and a guiding sheath 702 known to those of ordinary skill in the art. Dilator 704 has a tip 712 at the distal end and a proximal hub (not shown). Dilator 704 may have a radiopaque marker (not shown) located distally. Dilator 704 has a lumen (not shown) through which a guidewire (not shown) or the electrosurgical perforation device 100 can be delivered. Dilator 704 is most commonly delivered through the lumen (not shown) of a guiding sheath 702. Guiding sheath 702 has a tip 718 at the distal end and a proximal hub (not shown). Guiding sheath 702 may have a radiopaque marker (not shown) located distally.

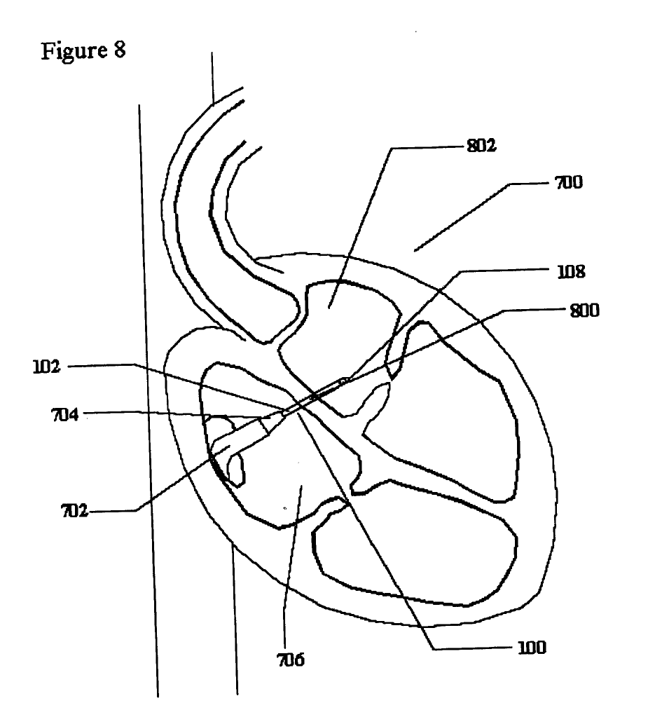

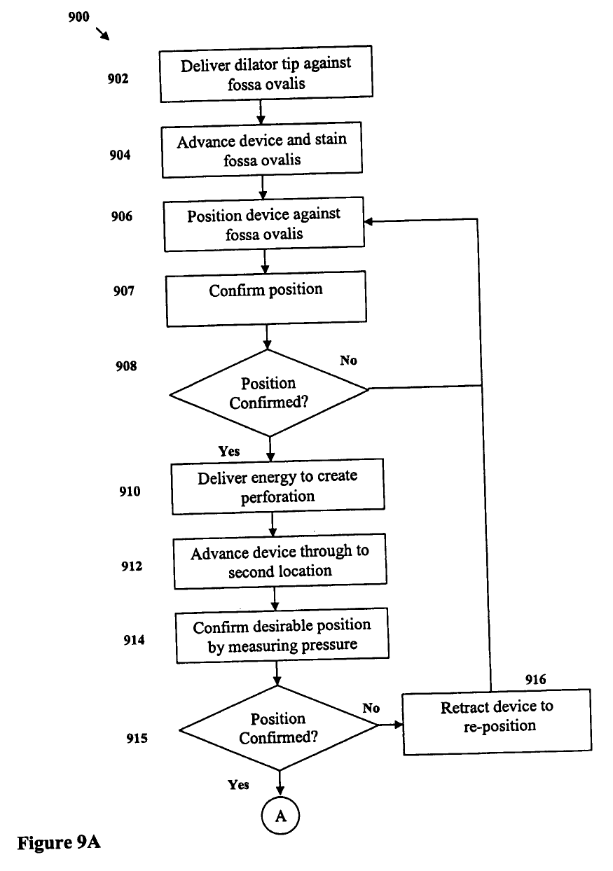

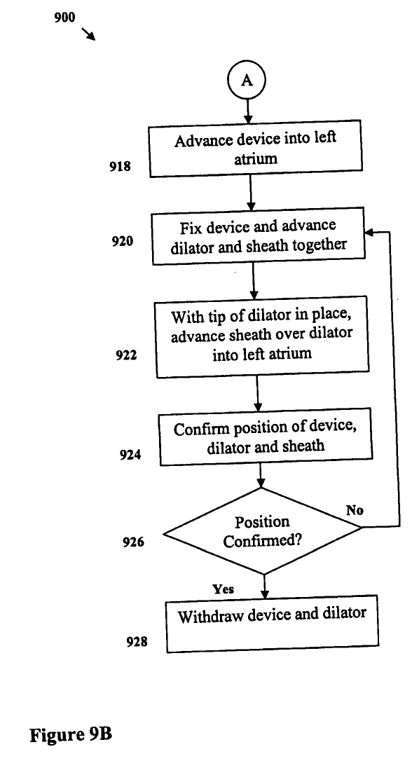

Referring to FIGS. 7 and 8 there is illustrated electrosurgical perforation device 100 inserted through a dilator 704 and sheath 702 within a heart 700 of a patient. A method 900 for creating a transseptal perforation is outlined in flow chart form in FIGS. 9A and 9B. In accordance with a method aspect of the invention for creating a transseptal perforation, to deliver the tip 712 of the dilator 704 against the fossa ovalis 710 (step 902) a guiding sheath 702 and dilator 704 with a lumen larger than the outer diameter of the electrosurgical perforation device 100 is introduced into a patient's vasculature. Guiding sheath 702 and dilator 704, known to those of ordinary skill in the art, are advanced together through the vasculature, approaching the heart from the Inferior Vena Cava 709, into the Superior Vena Cava (SVC) 707 of the heart 700. The sheath 702 and dilator 704 are withdrawn from the SVC, into a right atrium 706, and contrast agent is delivered through dilator 704 while dragging the dilator 704 and sheath 702 along an atrial septum 708 into a region of the fossa ovalis 710. The sheath 702 and dilator 704 are now positioned within the right atrium 706 of heart 700 so that the tip 712 of dilator 704 is located against the region of the fossa ovalis 710 on the atrial septum 708 (step 902).

Once the tip 712 of dilator 704 is in position against the region of the fossa ovalis 710, device 100 can be advanced through the dilator 704 until the functional tip 108 of device 100 is approximately lcm (about 0.39″) proximal to the tip 712 of dilator 704. Contrast agent delivered through device 100 will be directed through the tip 712 of dilator 704 directly into the tissue of the fossa ovalis 710, staining it radiopaquely (step 904). Under fluoroscopy, the stained region of the fossa ovalis 710 can be seen as a dark patch on a lighter gray colored atrial septum 708. Functional tip 108 is now easily directed toward the fossa ovalis 710, a preferred first desired location on the atrial septum 708 to create a perforation (FIG. 7 and step 906 of FIG. 9A).

In an alternate method, (not shown), once the tip 712 of dilator 704 is in position against the region of the fossa ovalis 710, contrast agent can be delivered through dilator 704 directly into the tissue of the fossa ovalis 710, staining it radiopaquely. Device 100 can now be advanced through dilator 704 while maintaining the position of tip 712 of dilator 704 against the fossa ovalis 710. Functional tip 108 can now easily be directed toward the fossa ovalis 710.

The position of device 100 may be confirmed by monitoring pressure at the functional tip 108 (step 907). Device 100 is coupled to external pressure transducer 118 and a right atrial pressure contour, known to those of ordinary skill in the art, may be shown on monitoring system 120. The technique for obtaining a pressure contour was previously described. The position of functional tip 108 may be additionally confirmed using an imaging modality such as fluoroscopy. Under fluoroscopy the radiopaque markings (not shown) associated with distal region 104 of device 100 may be aligned with the radiopaque marker (not shown) located distally on dilator 704 such that functional tip 108 of device 100 is located at the fossa ovalis 710. Alternately, the radiopaque markings (not shown) associated with distal region 104 of device 100 may be aligned with the radiopaque marker (not shown) located distally on sheath 702 such that functional tip 108 of device 100 is located at the fossa ovalis 710 (step 907). The position is evaluated and if the desired position is not confirmed (step 908, No branch), step 906 may be repeated. If confirmed (step 908, Yes branch), energy may be delivered to create the perforation. For example, generator 122 is activated and RF energy is delivered through device 100 to make a perforation 800 (step 910).

The functional tip 108 of device 100 is thereafter advanced through perforation 800 and into a second location (step 912). Advancement may be monitored under fluoroscopy using the radiopaque markings (not shown) on the distal region 104 of device 100. The preferred second location is left atrium 802 of the heart. The distal region 104 of device 100 is advanced incrementally into the left atrium 802 through dilator 704, for example, in 1 cm (about 0.39″) increments. The position of depth markings 117 of device 100 relative to proximal hub 714 of the dilator 704 can be used as a guide. Additionally, advancement of perforating device 100 can be controlled by monitoring the radiopaque markings on the distal region 104 of device 100 under fluoroscopy. When all openings 110 on distal region 104 of device 100 are located in the left atrium 802, the evaluation of the pressure contours from the pressure transducer (step 914) can be performed. Device 100 remains coupled to external pressure transducer 118 so that a pressure contour at the second location can be monitored.

After successful perforation a left atrial pressure contour, known to those of ordinary skill in the art, will be shown on the monitoring system. In the event that the imaging and pressure readings show that the perforation 800 is made in an undesirable location (step 915, No branch), device 100 is retracted into the right atrium 706 (step 916) and is repositioned for another perforation attempt (step 906). If perforation 800 is successfully made in the correct location (step 915, Yes branch), distal region 104 of device 100 is preferably further advanced through perforation 800. When device 100 is fully inserted into the dilator 704, housing 216 of the device 100 will be flush against proximal hub of the dilator 704, and no depth markings 117 of device 100 will be visible (step 918, FIG. 9B). When fully inserted, device 100 provides sufficient support to permit the dilator 704 to be advanced over it through perforation 800.

Housing 216 of device 100 may be fixed in place spatially, and both the proximal hub of dilator 704 and proximal hub of sheath 702 are incrementally advanced forward, together, thus sliding the dilator 704 and sheath 702 over device 100 (step 920). The tip 712 of dilator 704 and the tip 718 of sheath 702 are monitored under fluoroscopy as they are advanced over device 100 and once the tip 712 of dilator 704 has breeched the perforation 800, and advanced into the left atrium 802, the tip 718 of sheath 702 is advanced over dilator 704, across the perforation 800 and into the left atrium 802 (step 922).

In an alternate method of advancing the sheath and dilator into the left atrium, (not shown), once distal region 104 is fully advanced through perforation 800 and into the left atrium 802, and housing 216 of device 100 is flush against proximal hub of dilator 704, and no depth markings 117 of device 100 are visible, housing 216 of device 100, proximal hub of dilator 704 and proximal hub of sheath 702 may all be advanced forward together under fluoroscopy. Forward momentum will cause the tip 712 of dilator 704 to breech the perforation 800, advancing into the left atrium 802. The tip 718 of sheath 702 will follow over dilator 704, across the perforation 800 and into the left atrium 802.

At step 924, the positions of distal region 104 of device 100, tip 712 of dilator 704 and tip 718 of sheath 702 are confirmed, for example, under fluoroscopy to be in the left atrium 802. If not in the desired location (step 926), step 920 may be repeated. If the positions are confirmed (step 926), device 100 and dilator 704 may now be respectively withdrawn outside the body, preferably under fluoroscopic guidance (step 928). While maintaining the position of tip 712 of dilator 704 and tip 718 of sheath 702 in the left atrium 802, device 100 may be withdrawn. Dilator 704 may now be withdrawn outside the body under fluoroscopic guidance, while maintaining the position of the tip 718 of sheath 702 in the left atrium 802. Optionally, a contrast agent may now be injected through sheath 702 into the left atrium 802, or blood aspirated through sheath 702 from the left atrium 802 and sheath 702 may now be used to deliver other catheters (not shown) to the left atrium 802.

The present invention in various aspects thus provides a device and method that is capable of creating a controlled perforation while determining a position of the device in response to pressure at a location in the body. The present invention also provides a method for staining the area to be perforated in order to make it easier to locate during the perforation. In addition, the present invention provides a method for delivering a dilator and sheath over the device after the perforation. The controlled perforation is created by the application of energy by a generator to a functional tip on the device. A means for determining the position of the device may comprise a pressure transmitting lumen that can be releasably coupled to an external pressure transducer. In this embodiment, there is at least one opening near the distal region of the device for blood or other fluid to enter and fill the lumen and exert a measurable pressure on a coupled external transducer. The lumen and opening may also be useful for injecting radiopaque contrast or other agents through the device. In an alternate embodiment, the means for determining a position of the device in response to pressure comprises a transducer located on the device proximal to the functional tip.

The device of the invention is useful as a substitute for a traditional transseptal needle to create a transseptal perforation. The device of the present invention preferably has a soft distal region with a functional tip that uses RF energy to create a perforation across a septum, making the procedure more easily controlled and less operator dependent than a transseptal needle procedure. The soft distal region of the device reduces incidents of vascular trauma as the device is advanced through the vasculature. The application of RF energy is controlled via an electric generator, eliminating the need for the operator to subjectively manage the amount of force necessary to cross the septum with a traditional needle. The present invention eliminates the danger of applying too much mechanical force and injuring the posterior wall of the heart.

The present invention also provides a method for the creation of a perforation in an atrial septum. Pressure monitoring is particularly important in this procedure, as there is the possibility of inadvertently perforating the aorta due to its proximity to the atrial septum. Pressure measurements allow the operator to confirm that the distal end of the device has entered the left atrium, and not the aorta, or another undesirable location in the heart. Staining the atrial septum is also particularly important in this procedure, as it easily identifies the region of the atrial septum (fossa ovalis) to be perforated. Preferably, the device will also be visible using standard imaging techniques; however the ability to monitor pressure provides the operator with a level of safety and confidence that would not exist using only these techniques.

The present invention also provides a method for delivering the dilator and sheath over the electrosurgical perforation device into the left atrium once a successful perforation has been created. One of the main reasons for creating a transseptal perforation is to gain access to the left side of the heart for delivery of catheters or devices to treat left-sided heart arrhythmias or defects.

While the surgical device thus described is capable of cutting living tissue, it will be understood by persons of ordinary skill in the art that an appropriate device in accordance with the invention will be capable of cutting or removing material such as plaque or thrombotic occlusions from diseased vessels as well.

Although the above description relates to specific embodiments as presently contemplated by the inventors, it is understood that the invention in its broad aspect includes mechanical and functional equivalents of the elements described herein.

What is claimed is:

1. A surgical device for cutting material and monitoring pressure comprising:
an elongate member having a distal region and a proximal region;
an energy delivery device associated with the elongate member at the distal region for delivering cutting energy to the material, said energy delivery device adapted for connection to an energy source; and
a pressure sensing mechanism associated with the distal region for monitoring pressure about the distal region.

2. The device as claimed in claim 1 wherein the cutting energy is at least one form of energy selected from a group consisting of: electrical current; microwave; ultrasound;
and laser.

3. The device as claimed in claim 2 wherein the electrical current has a frequency within the radio frequency range.

4. The device as claimed in claim 1 wherein the material comprises cellular tissue and wherein the energy delivery device is operable to deliver sufficient energy to the tissue to result in a rapid increase in the intracellular temperature causing vaporization of intracellular water and subsequent cell lysis.

5. The device as claimed in claim 1 wherein the pressure sensing mechanism comprises a pressure transmitting lumen extending between the proximal and distal regions, said lumen at the proximal region being adapted for fluid communication with a pressure transducer that provides a signal which varies as a function of pressure and adapted at the distal region for fluid communication with an environment about said distal region.

6. The device as claimed in claim 5 wherein the distal region comprises at least one opening to the environment and wherein the lumen is in fluid communication with the at least one opening.

7. The device as claimed in claim 6 wherein the lumen is adapted for injecting a fluid through the at least one opening.

8. The device as claimed in claim 5 wherein the distal region comprises multiple openings to the environment and wherein the lumen is in fluid communication with the multiple openings.

9. The device as claimed in claim 8 wherein at least some of the multiple openings are located distally and some of the multiple openings are located proximally with respect to each other and wherein the some of the openings located distally are larger than the some of the openings located proximally.

10. The device as claimed in claim 8 wherein the lumen is adapted for injecting a fluid through the multiple openings.

11. The device as claimed in claim 1 wherein the pressure sensing mechanism comprises a pressure transducer on-board the elongate member, said transducer being adapted for communication with a pressure monitoring system.

12. The device as claimed in claim 1 wherein the energy delivery device comprises a functional tip with at least one active electrode.

13. The device as claimed in claim 1 wherein the energy delivery device comprises a functional tip having two or more electrodes.

14. The device as claimed in claim 13 wherein the electrodes are configured in an arrangement where at least one of the electrodes is active and at least one is a return electrode.

15. The device as claimed in claim 1 comprising at least one depth marking.

16. The device as claimed in claim 1 comprising at least one radiopaque marker.

17. The device as claimed in claim 1 comprising a radiopaque distal region.

18. A method of surgery comprising the steps of:
(i) introducing a surgical device into a body of a patient, the surgical device comprising an elongate member having a distal region and a proximal region, an energy delivery device proximate to the distal region capable of cutting material and a pressure sensing mechanism for determining pressure in the body proximate to the distal region;
(ii) positioning the energy delivery device to a first desired location in the patient's body adjacent material to be cut;
(iii) delivering energy using the energy delivery device to cut said material; and
(iv) measuring pressure in the body using the pressure sensing mechanism in order to determine the position of the surgical device at least one of before and after step (iii).

19. The method as claimed in claim 18 wherein step (ii) comprises staining a region of tissue in the first desired location in the patient's body.

20. The method as claimed in claim 18 further comprising a step of:
(v) advancing the device to a second desired location.

21. The method as claimed in claim 20 wherein the surgical device comprises at least one depth marking and at least one radiopaque marker and wherein step (v) comprises monitoring at least one of said depth markings and at least one of said radiopaque markers.

22. The method as claimed in claim 20 further comprising a step of:
(vi) measuring pressure using the pressure sensing mechanism at the second location.

23. The method as claimed in claim 22 wherein the surgical device comprises at least one depth marking and at least one radiopaque marker and wherein step (vi) is performed after confirming the position of the pressure sensing mechanism at the second location using at least one of said depth markings and said radiopaque markers.

24. The method as claimed in claim 18 wherein step (i) comprises introducing the device into the patient's vasculature.

25. The method as claimed in claim 24 wherein the step of introducing the device into the patient's vasculature comprises inserting the device into a dilator and a guiding sheath positioned in the patient's vasculature.

26. The method as in claim 25 wherein the device and at least one of the dilator and sheath comprise a radiopaque marking and wherein step (ii) comprises aligning the radiopaque markings to aid in positioning the device.

27. The method as claimed in claim 25 comprising a step of:
(v) advancing the dilator and the sheath into the second location together over the spatially fixed surgical device.

28. The method as claimed in claim 25 comprising a step of:
(v) advancing the dilator, sheath and surgical device all together into the second location.

29. The method as claimed in claim 18 wherein the material is tissue located on an atrial septum of a heart.

30. The method as claimed in claim 19 wherein the region of tissue to be stained is a fossa ovalis of a heart.

31. The method as claimed in claim 22 wherein the pressure measured at the second location is the blood pressure in the left atrium.

32. An electrosurgical device comprising:
a elongate member having a distal region and a proximal region, said distal region insertable within and along a lumen within a body of a patient and maneuverable therethrough to a desired location where the device is operated to cut material and monitor pressure at the desired location;
at least one electrode associated with the distal region for cutting tissue, said at least one electrode adapted for coupling to an electrical power source; and
a pressure sensing mechanism associated with the distal region for sensing pressure at the desired location within the body, said mechanism adapted for coupling to a pressure monitoring system.

33. The device as claimed in claim 32 wherein the pressure sensing mechanism is configured to minimize a portion of the elongate member that is necessary to be located at the desired location to monitor pressure.

34. The device as claimed in claim 32 wherein the pressure sensing mechanism comprises a pressure transmitting lumen defined within the elongate member extending from the proximal region to and through at least one opening defined in the distal region.

35. The device as claimed in claim 34 wherein said proximal region is adapted for coupling said pressure transmitting lumen to a pressure transducer associated with the pressure monitoring system.

36. The device as claimed in claim 34 wherein the pressure transmitting lumen is adapted for at least one of injecting a fluid to or removing a fluid from said body.

37. The device as claimed in claim 34 wherein the at least one electrode is coupled to said energy source by a coupling means extending through said pressure transmitting lumen.

38. The device as claimed in claim 32 wherein said pressure sensing mechanism comprises an on-board pressure transducer adapted for communicating a transduced pressure signal representative of pressure about the distal region to said pressure monitoring system.

39. The device as claimed in claim 32 wherein the at least one electrode defines a functional tip comprising a conductive and radiopaque material at said distal region.

40. The device as claimed in claim 39 wherein the electrical power source is capable of providing a high-frequency electrical power to said functional tip in a high impedance range.

41. The device as claimed in claim 32 wherein the proximal region is adapted to releasably couple said pressure sensing mechanism to said pressure monitoring system.

42. The device as claimed in claim 32 wherein the proximal region is adapted to releasably couple said electrode to said electrical power source.

43. A surgical device comprising:
means for cutting material at a desired location in a body of a patient; and
means for determining a position of the device responsive to pressure within the body.

44. The device as claimed in claim 43 comprising a flexible elongate member having a proximal region and a distal region, said distal region adapted for insertion within and along a lumen within the body and maneuverable therethrough to the desired location; and wherein said means for determining a position of the device is operable to determine the position of the distal region.

45. The device as claimed in claim 43 wherein the means for determining a position of the device comprises a pressure transducer for providing a signal representative of pressure to a pressure monitoring system.

46. The device as claimed in claim 45 wherein the means for determining a position of the device comprises a pressure transmitting lumen defined by the device for coupling to the pressure transducer.

47. The device as claimed in claim 46 comprising a means for injecting fluid to and removing fluid from the body.

48. A method of cutting tissue at a desired location in a body of a patient comprising the steps of:
inserting a surgical device into the body, said surgical device comprising means for cutting material and means for determining a position of the device responsive to pressure within the body; and
positioning said surgical device at the desired location in response to the means for determining a position of the device.

49. The method as claimed in claim 48 comprising the step of:
cutting material at the desired location.

50. The method as claimed in claim 49 comprising the step of:
advancing said device in the body in response to said means for determining a position of the device.

51. The method as claimed in claim 50 comprising re-positioning said device for re-cutting in response to said means for determining a position of the device.
